(19) United States
(12) Patent Application Publication
(10) Pub. No.: US 2009/0124863 A1
(43) Pub. Date: May 14, 2009

(54) METHOD AND SYSTEM FOR RECORDING PATIENT-STATUS

(75) Inventors: Alan Liu, Bartlett, IL (US); David Phillip Murawski, Cary, IL (US); Murali Kumaran Kariathungal, Hoffman Estates, IL (US)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 11/936,874

(22) Filed: Nov. 8, 2007

(51) Int. Cl. A61B 5/00

(52) U.S. Cl. 600/300

(57) ABSTRACT

A method and system for recording patient status automatically is disclosed herein. The method of recording patient-status comprises: monitoring the gestures of a patient continuously using a video imager. From the monitored gestures a patient-status corresponding to at least one clinical factor is identified and the patient-status is automatically recorded in an electronic medical record (EMR). The method and system describes an exemplary embodiment of determining pain level of a patient in a clinical environment using facial expressions and sound.

### FIELD OF THE INVENTION

This invention relates generally to patient monitoring, and more particularly to, a method and system for automatically recording a patient-status.

### BACKGROUND OF THE INVENTION

Monitoring clinical status, generally referred to as patient-status, is very important in clinical environments, especially when the patient is not able to effectively communicate his or her status to a caretaker. The patient-status needs to be monitored continuously as it can change at any time. Generally the caretaker manually monitors different patient-statuses such as normal, danger, alert, etc. with reference to patient's various clinical factors such as pain level, tension, uneasiness, etc. The caretaker typically examines the patient at a preset interval for checking the patient-status and records the patient-status along with corresponding critical factors in a follow-up sheet manually. This process is subject to errors as the caretaker may fail to monitor or record the patient-status at the preset intervals or the patient-status may vary considerably at a time when the patient is not scheduled for status monitoring.

An example of patient-status is the pain level experienced by a patient while he is under clinical observation. Measuring pain in clinical workflows is very difficult as the feeling of pain is subjective. In traditional clinical workflows, the caretaker maintains flow sheets for recording the pain level of each patent. The flow sheet is updated on a preset interval by collecting pain related information from the patient.

However there are instances where a patient cannot communicate his or her pain level effectively to the caretaker. For examples, infants, elderly patients, patients in coma, patients under anaesthesia, etc. may not be able to communicate the pain level effectively to the caretaker. The patient is also generally not able to communicate its pain level effectively to a caretaker in veterinary applications where the patient is an animal such as a dog, cat, horse, cow, sheep, mouse or other non-human being.

Generally the pain level is monitored regularly, but not continuously. The caretaker records the pain level at regular intervals and in some instances may forget to observe or record the pain level. This may restrain the caretaker from giving appropriate care to the patient. Manual monitoring of the pain continuously is not normally feasible or practical and is cumbersome.

As the caretaker is monitoring the pain at regular intervals, there is a chance that the caretaker may not monitor or observe the pain level when the patient is having acute or severe pain. Hence it would be beneficial to provide a mechanism to alert the caretaker automatically whenever the patient suffers from acute pain.

Even though there are several methods to measure pain level of a patient, many of them are dependent on the information collected from the patients in periodic intervals. Further the feeling of pain is subjective and hence maintaining a general standard or scale to identify the pain level may not be accurate.

Thus there exists a need to provide an objective and automated method for monitoring and recording patient status including pain level of a patient, in real time.

### SUMMARY OF THE INVENTION

The above-mentioned shortcomings, disadvantages and problems are addressed herein which will be understood by reading and understanding the following specification.

One embodiment of the present invention provides a method of recording patient-status. The method comprises: monitoring the gestures of a patient continuously using a video imager and identifying the patient-status corresponding to at least one clinical factor using the at least one monitored gesture. The method further includes recording the patient-status automatically in an electronic medical record (EMR).

In another embodiment, a method for determining pain level in a clinical environment is disclosed. The method comprises: monitoring continuously at least facial expressions and sound generated by a patient; analyzing the facial expressions and sound for determining the pain level; and translating the identified facial expressions and sound to a quantifiable parameter indicating the pain level. The method may further include automatically recording the pain level of a patient at a given instance.

In yet another embodiment, an automatic pain recording system is disclosed. The system comprises: a detector for continuously monitoring gestures of a patient including facial expressions and sound generated by the patient; an analyzer coupled to the detector for analyzing the gestures for identifying a pain level; and a recorder for recording the identified pain level.

Various other features, objects, and advantages of the invention will be made apparent to those skilled in the art from the accompanying drawings and detailed description thereof.

### BRIEF DESCRIPTION OF THE DRAWINGS

FIG. 1 is a flowchart illustrating a method of automatically recording patient-status as described in an embodiment of the invention;

FIG. 2 is a detailed flowchart illustrating an automated patient-status recording method as described in an embodiment of the invention;

FIG. 3 is a block diagram of an automated patient-status recording system as described in an embodiment of the invention;

FIG. 4 is a detailed block diagram of an automated patient-status recording system as described in an embodiment of the invention;

FIG. 5 is a flowchart illustrating a method of determining pain-level as described in an embodiment of the invention; and

FIG. 6 is a block diagram of a pain recording system as described in an embodiment of the invention;

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

In various embodiments, a method and system for automatic patient-status recording is disclosed. The patient-status is monitored continuously and is recorded automatically. The method includes monitoring at least one gesture of a patient continuously and, based on the gestures, deriving a patient-status corresponding to a clinical factor at any given time. In an embodiment, the system incorporates a video imager for recording the images along with the sound generated by the patient.

In an embodiment, the patient-status corresponding to various clinical factors are identified. The non-limiting examples of clinical factors include pain level, tension level, anxiety, uneasiness, blood pressure, fear etc and the example of patient-status may include “Normal”, “Alert”, “Caution”, “Serious”, “Severe”, “Danger” or “Critical”. The patient-status is recorded along with the clinical factor.

In an embodiment, the patient-status may be used to trigger an alarm to notify the caretaker or the doctor to provide immediate attention to the patient.

In an embodiment, the method translates different gestures to a quantifiable parameter, so that it can be recorded in a uniform format.

In an embodiment, the patient-status at a given instance is updated in an electronic medical record (EMR).

In an embodiment, the patient-status is derived in real time using facial expressions and the sound generated by the patient.

In an embodiment, the invention discloses a method and system for automatically determining and recording pain-level of a patient. In an example, the pain-level is ascertained using facial expressions and the sound generated by the patient.

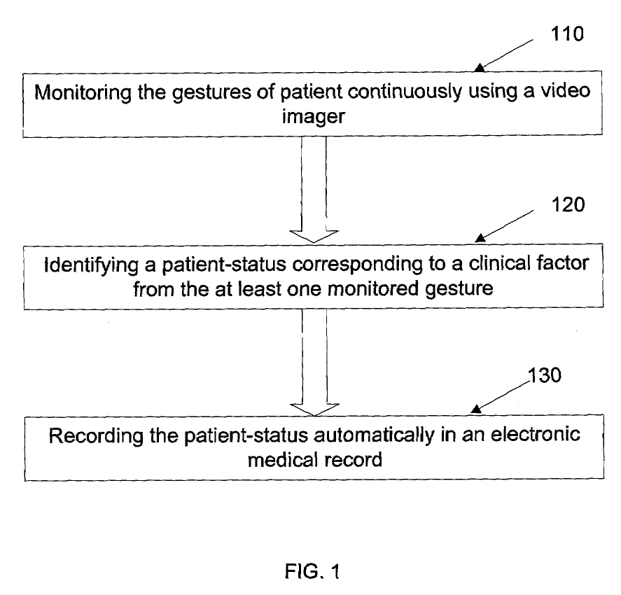

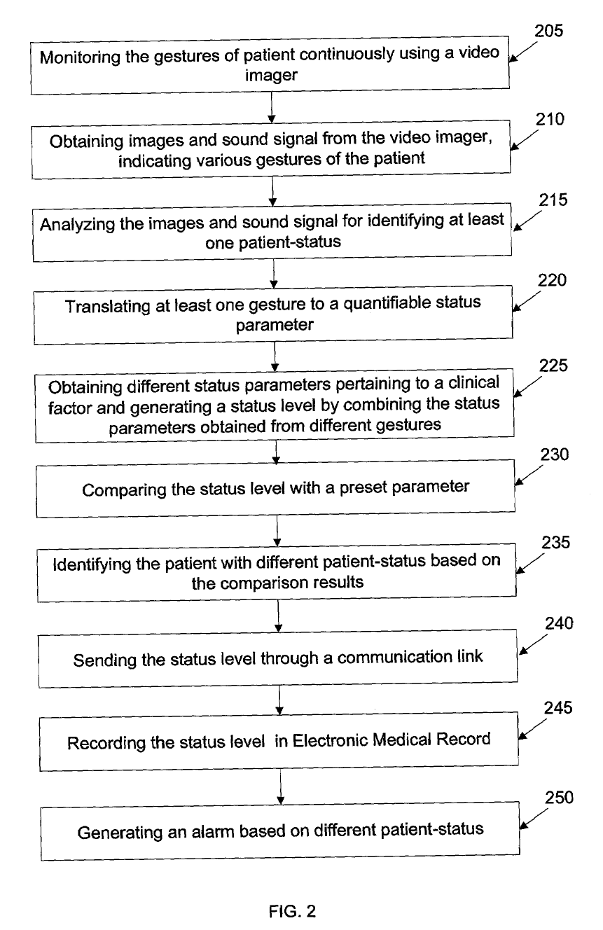

FIG. 1 is a flowchart illustrating an automated patient-status recording method as described in an embodiment of the invention. At step 110, various gestures of a patient are monitored using a video imager. The gestures are monitored continuously. Some of the examples of gestures include facial expressions, sound, movements of body parts, activity, cry, and consolability of the patient, however the examples need not be limited to these. At step 120, a patient-status corresponding to at least one clinical factor is identified from the at least one monitored gesture. For example, the gesture facial expressions may be linked with clinical factors such as pain level of the patient or the gesture sound generated by the patient may be associated with a clinical factor such as tension level. Various gestures may commonly define a patient-status corresponding to a clinical factor, for example, for determining the pain level, facial expressions of the patient and sound generated by the patient may be considered. Also same gestures may be used to define patient-status corresponding to different clinical factors, for example sound generated by patient may be used in defining the pain level as well as the tension level. For defining the patient-status, the monitored gestures are analyzed. In an example, the gestures are obtained in the form of video images, which include sound signals as well. Once the images and sound signals are obtained, they are analyzed and converted into a status parameter, a quantified parameter such as a numerical value indicating the patient-status values. There could be different gestures monitored for defining patient-status corresponding to a clinical factor and the status parameter from each gesture can be combined to define a single status level. In an embodiment, the status level can be compared with a preset parameter, to assign different patient-status to the patient. For example, a threshold value can be set for patient and, based on comparison of the status level with the threshold value; different patient-status such as “Normal”, “Alert”, “Danger”, etc. can be assigned. At step 130, the obtained status-level is recorded automatically in an electronic medical record (EMR) along with the corresponding clinical factors. The patient-status can also be recorded along with the status-level. The various steps involved in the method are explained in detail in FIG. 2

FIG. 2 is a detailed flowchart illustrating an automated patient-status recording method as described in an embodiment of the invention. At step 205, different gestures of a patient are monitored using a video imager. The video imager records images and the sound signal. The sound signal includes sound generated by the patient due to different clinical factors such as pain, tension, fear, etc. Different gestures could include facial expressions, sound, and movements of body parts, activity, cry, and consolability of the patient. At step 210, the images and sound signal in the form of video signal and audio signal are obtained from the video imager. At step 215, the images and sound signal are analyzed for identifying a patient-status corresponding to at least one clinical factor. The authenticity of images and audio signal are verified. Different features or parameters of the gesture may be analyzed for deriving patient-status corresponding to different clinical factors. For example, in identifying the pain level, the chin movements from the monitored facial expression of the patient may be analyzed and on the other hand for identifying the tension level, the eyeball movements of the patient may be analyzed. These are examples for illustration and need not be considered as limiting. The analyzer identifies the relevant gestures corresponding to the clinical factor and analyzes the identified gestures. At step 220, at least one gesture is translated to a status parameter. For example, if the patient-status corresponding to a clinical factor such as patient's pain level needs to be analyzed, at lest one gesture from the images such as facial expressions of patient is considered and is analyzed. The facial expressions noticed from the images need to be converted to a numerical or quantifiable parameter that will represent status value such as pain-level, expressed as the status parameter. So translation techniques are used to convert the facial expressions to a numerical value. At step 225, the status parameters pertaining to a clinical factor are obtained from different gestures and are combined. The combined parameter referred to as status level will indicate the status parameter corresponding to a clinical factor. Similarly status level corresponding to different clinical factors can be obtained. At step 230, the status level is compared with a preset parameter. The preset parameter can be a threshold value pertaining to different patient-status such as tolerable pain level; safe tension level, etc. and the threshold level could be set depending on the patient, clinical situation, etc. Based on the comparison result, different patient-status can be assigned as indicated at step 235. The comparison of status level with the threshold value will help in assigning the patient with different patient-status. For example, if the status level is higher than the threshold value, then the patient is assigned with a patient-status as “Critical”, hence needs immediate attention and this can be conveyed to the caretaker appropriately. Based on the comparison results, the patient can be assigned with different patient-status such as “Normal”, “Alert”, “Caution”, “Serious”, “Severe”, “Danger” or “Critical”. At step 240, the status level is send through an electronic link to a destination where EMR is located. If required the corresponding clinical factor may also be sent along with the status level. At step 245, the status level is recorded in EMR along with the corresponding clinical factor. The patient-status corresponding to different clinical factors can also be recorded in the EMR. At step 250 an alarm is generated based on the patient-status and will help in providing appropriate care to the patients. Different forms of alarms can be selected based on different clinical factors and/or patient status. For example, if the patient status is critical the alarm may generate a loud noise and if it is just an advice it may display the message without making any noise. Similarly different colors may be used for displaying patient status based on the nature of the patient status.

Even though the method explained above refers to monitoring and recording of patient-status corresponding to one clinical factor at a time, the application of the method need not be limited to one clinical factor. A plurality patient-status corresponding to different clinical factors can be monitored from different gestures at the same time and can be analyzed and recorded simultaneously. For example, from facial expression and sound generated by the patient, different clinical factors such as pain level, fear level, etc. can be analyzed and corresponding patient-status can be assigned. Alternately, different gestures and/or same gestures may be analyzed for identifying different patient-status corresponding to different clinical factors.

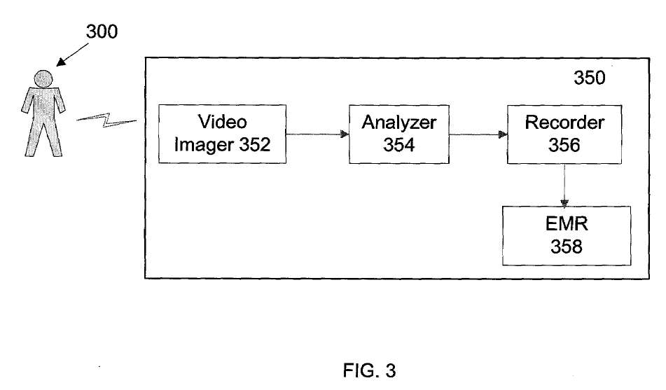

FIG. 3 is a block diagram of an automated patient-status recording system as described in an embodiment of the invention. The patient-status recording system 350 is configured to monitor and record patient-status corresponding to different clinical factors such as pain level, tension level, etc. of a patient 300 using different gestures of the patient 300. Different gestures could include facial expressions, sound, and movements of body parts, activity, cry, and consolability of the patient 300. The gestures could be monitored continuously and could be limited to particular body parts such as the face. In alternative embodiments the gestures can be monitored with respect to the whole body such as patient movements, activities, etc. The patient-status monitoring system 350 includes a video imager 352, an analyzer 354, a recorder 356 and an Electronic Medical Record (EMR) 358. The video imager 352 is configured to monitor the patient 300 continuously for recording the gestures. In an embodiment the video imager 352 may record only the facial expressions and sound generated by the patient. The video imager 352 records images and the sound signal and the sound signal may include the sound generated by the patient due to pain or fear or any other relevant factors. The video imager 352 is coupled to the analyzer 354 and feeds the images and the sound signal to the analyzer 354. The analyzer 354 analyzes the images and sound signal for analyzing various gestures of the patient and derives a status level corresponding to a clinical factor, from the gestures. The analyzer 354 checks the authenticity of the images and the sound signal and identifies the relevant gestures pertaining to a clinical factor. The analyzer 354 coverts different forms of analyzed information such as image or sound signal to a status-parameter that defines the values of the status. Different status parameters obtained from different gestures corresponding to a clinical factor is combined and a status level is generated. Once the analyzer 354 defines the status level it may assign different patient-status to the patient 300, using results of comparison of the status level with a threshold value. The status level is recorded automatically to appropriate medium using a recorder 356. In an example, the recorder 356 records the patient-status and/or the status level into an EMR 358 along with the corresponding clinical factor. Thus the patient-status recording system 350 facilitates automatically updating EMR 358 with the patient-status.

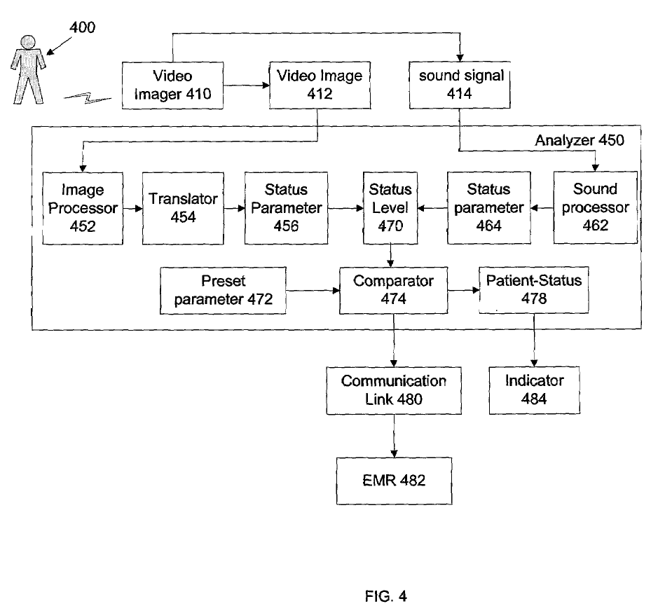

FIG. 4 is a detailed block diagram of an automated patient-status monitoring system as described in an embodiment of the invention. The patient 400 is monitored by a three dimensional imager continuously. In an example the three-dimensional imager is a video imager 410. The video imager 410 monitors the patient and records various gestures of the patient 400. The gestures are recorded in the form of video images 412, hereinafter referred to as images, and sound signal 414. The images 412 reveal various gestures of the patient such as facial expressions, body movements, activities, etc. and the sound signal 414 will capture the sound generated by the patient 400 due to various clinical factors such as pain or fear. The image 412 and sound signal 414 are fed to an analyzer 450 for analyzing them. The analyzer 450 analyzes the images 412 and sound signal 414 separately. The images 412 are fed to an image processor 452 and the images 412 are analyzed for identifying relevant gestures. In an example facial expressions from the images are analyzed. However the images may be analyzed for identifying patient movements, activity levels, etc. Even from the facial expressions, the facial expressions relevant in determining a patient-status pertaining to a particular clinical parameter such as pain level may be selected for analysis. Once the relevant gestures are identified, the translator 454 coupled to the image processor 452, translates the gestures identified from the images 412 to a quantifiable status parameter 456 indicating the status value of the patient at a given instance. Similarly the sound signal 414 is fed to a sound processor 462 and the sound generated due to pain is identified from the sound signal 414 and analyzed for identifying a status parameter 464 indicating status value at a given time. The status parameters 456 derived from the images 412 and the status parameters 464 derived from the sound signal 414 are combined together to derive a status level 470 of the patient. The status level 470 conveys numerical value of the status corresponding to a particular clinical factor.

From the status level 470, different patient-status 478 can be derived. A preset parameter 472 indicating a threshold value of a clinical factor may be defined by a clinician or a caretaker corresponding to the patient. The status level 470 can be compared with the preset parameter 472 using a comparator 474. Based on the comparison results different patient-status 478 can be assigned to the patient 400. For example, if the status level 470 is very high compared with the preset parameter 472, the patient 400 may be assigned with a patient-status 478 as “Danger”. The status level 470 and/or the patient-status 478 can be sent to an Electronic Medical Record (EMR) 482 through a communication link 480 along with the corresponding clinical factor. The status level 470 or the patient-status 478 can be recorded in the EMR 482. Further the patient-status 478 may be fed to an indicator 484 for indicating the patient-status 478 to the caretaker or to the clinician. This will ensure the prompt care of the patient 400 based on his status.

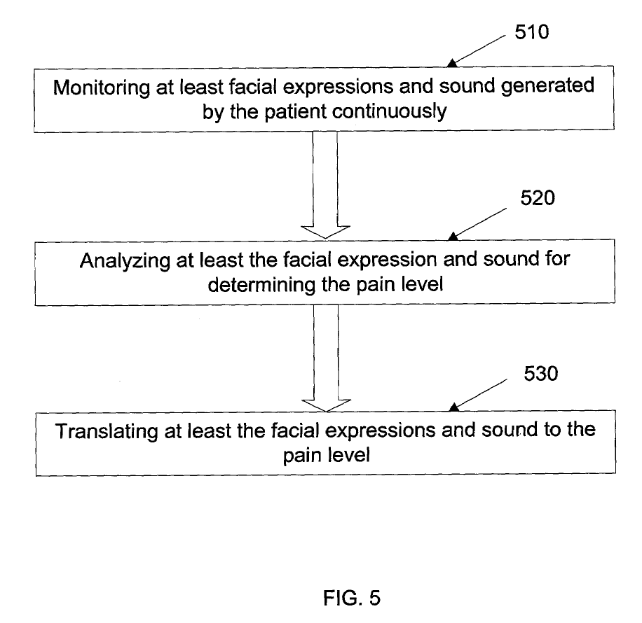

FIG. 5 is a flowchart illustrating a method of determining pain in a clinical environment as described in an embodiment of the invention. In an embodiment, the method further includes automatically recording clinical pain. The pain level is measured continuously without inquiring with the patient, in other words pain is measured objectively. The pain value is obtained from different gestures of the patient. In an example the pain level is determined from the facial expressions and sound generated by the patient due to pain. At step 510, patient's facial expression and sound generated by the patient are monitored continuously. For monitoring the facial expressions and sound, a three dimensional imager is provided. In an example, the facial expressions and sound generated by the patient can be recorded as images and sound signal using a video imager. At step 520, the facial expressions and the sound are analyzed for determining the pain level. The step identifies facial expressions and sound relevant in determining pain level. Different types of analysis such as lookup table method, analyzing changes in the various features such as eyeball movements of the patient, chin movement etc. However the techniques used in analysis need not be limited to these. The step further includes verifying the authenticity of the images and sound generated by the patient. In an example, the pain level can be obtained from the images or sound. At step 530, the facial expression and sound are translated to a quantifiable parameter, referred as pain-value corresponding to the value of pain. For example, there are some instances where patient is not capable of generating any sound and in this event only the facial expressions of the patient is considered for deriving the pain level. In the event where the facial expressions and sound are obtained, pain values from the facial expressions and sound are derived. The pain values derived using the facial expressions and sound is combined to form a pain level corresponding to a patient at a given instance. The pain level obtained can be compared with a threshold pain-value and different patient-status can be assigned based on the comparison result. For example, if the patient has undergone a surgery, the clinician may set the threshold pain-value at a certain level. After obtaining the pain level from facial expressions and/or sound, it can be compared with the threshold value. If the pain level is less than the threshold value the patient may be given with a patient-status as “normal” and if it is much higher than the threshold value, the patient can be assigned with a patient-status as “critical”. Based on comparison result of threshold value with the pain level, the patient can be assigned with different status such as “Normal”, “Alert”, “Danger”, “Critical” etc and based on the patient status the caretaker can take the appropriate action. The pain level obtained is recorded electronically. In an example the pain level obtained is recorded in an EMR. This step includes transmitting the pain level through a communication link, for recording the pain level in the EMR. The pain level can be stored in different mediums electronically so that human intervention in recording is kept at minimal. The method can include generating an alarm based on the different patient-status or different pain level. This will alert the caretaker to take necessary action based on the recorded pain level.

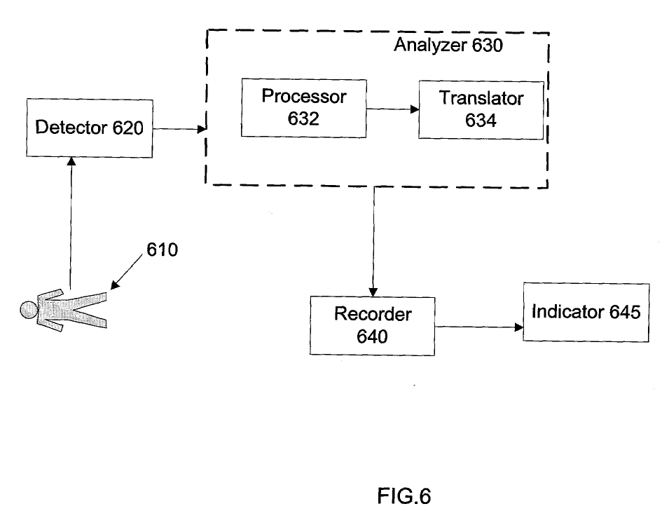

FIG. 6 is a block diagram of a pain recording system as described in an embodiment of the invention. A patient 610 having clinical pain is monitored using a detector 620. The detector 620 is a three dimensional imager configured to monitor the patient continuously. The detector 620 is located such that various gestures of the patient are captured appropriately for analyzing them. In an example the detector 620 is a video imager. The detector 620 monitors the patient continuously and records various gestures as images and sound signal. The images will identify various gestures of the patient 610 including the movements, body activity, facial expression, etc. and the sound signal will capture sound generated by the patient due to pain, tension or any other relevant factors. The detector 620 is coupled to an analyzer 630, wherein the images and sound are analyzed for deriving a pain level of the patient 610 at a given instance. The analyzer 630 includes a processor 632 and a translator 634. In an example the facial expressions and sound generated by the patient 610 are processed. The processor 632 identifies the relevant gestures that need to be analyzed corresponding to a clinical factor or a patient. For example if the patient is not able to generate any sound, the processor 632 will analyze only the images and will not consider sound for analysis. The gestures need to be analyzed for different clinical factors such as pain level, tension level etc will be different and the processor 632 identifies the relevant gestures. The translator 634 converts the facial expressions and sound to a numerical value indicating the pain-value. The analyzer 630 may use different analysis and translation techniques in deriving the pain-value from the facial expressions and sound. The pain-values obtained from the facial expressions and sound are combined to form a pain level indicting the overall pain-value of the patient. Once the pain level is obtained, a recorder 640 records the pain level in a flowchart electronically. In an example the flowchart could be an EMR. Further the processor 632 can compare the pain level with a preset threshold value and based on the comparison results, different patient-status such as “Normal”, “Alert”, “Caution”, “Serious”, “Severe”, “Danger” or “Critical” etc can be assigned to the patient. The recorder 640 may further include an indictor 645 for indicting a patient-status derived from the pain level to a caretaker. The patient-status is obtained by comparing the pain value with a threshold pain level.

The advantages of the invention include reduction of human errors in clinical workflows, especially in patient monitoring. The method increases the agility of the healthcare services, as the human intervention in recording the patient-status is minimum. Also since the patient-status is recorded in EMR, clinicians or physicians who are located at a distance from the patient can receive the patient-status and also this will help will help the physicians in analyzing the patient-status at a later stage based on the patient-status recorded in the EMR. Further monitoring the patient status continuously in real time and generating alarm based on the monitored status, helps in improving the clinical workflow. Thus the method and system will improve the patient care.

Thus various embodiments of the invention describe method and system for recording patient-status using various gestures of the patient. An exemplary embodiment of the invention provides a method f determining pain level of a patient using his facial expressions and sound generated by the patient.

While the invention has been described with reference to preferred embodiments, those skilled in the art will appreciate that certain substitutions, alterations and omissions may be made to the embodiments without departing from the spirit of the invention. Accordingly, the foregoing description is meant to be exemplary only, and should not limit the scope of the invention as set forth in the following claims.

We claim:

1. A method of recording patient-status, comprising:
monitoring the gestures of a patient continuously using a video imager;
identifying the patient-status corresponding to at least one clinical factor using at least one monitored gesture; and
recording the patient-status automatically in an electronic medical record (EMR).

2. A method as in claim 1, wherein the step of monitoring the gestures of a patient comprises: monitoring at least one of facial expressions, sound, movements of body parts, activity, cry, and consolability of the patient.

3. A method as in claim 1, wherein the step of identifying the patient-status comprises: analyzing images and sound obtained by the video imager.

4. A method as in claim 3, wherein the step of analyzing further comprises: translating the gestures of the patient to a status parameter, wherein the status parameter indicates values of the gesture.

5. A method as in claim 4, wherein the step of analyzing further comprises: generating a status level pertaining to a clinical factor using a plurality of status parameters derived from different gestures.

6. A method as in claim 4, wherein the step of analyzing further comprises: comparing the status level with a set of preset parameter for deriving a patient-status.

7. A method as in claim 5, wherein the step of analyzing further comprises: identifying the patient-status as normal, caution, alert, serious, severe, danger or critical based on the status level.

8. A method as in claim 5, wherein the method further comprises: defining different patient-status corresponding to different clinical factors such as pain, tension, blood pressure, anxiety, uneasiness or fear.

9. A method as in claim 1, wherein the step of recording comprises: electronically recording the status level along with the corresponding clinical factor in an electronic medical record (EMR).

10. A method as in claim 1, wherein the step of recording further comprises: sending the status level through a communication link to record the status level in an electronic medical record (EMR).

11. A method as in claim 7, wherein the step of recording further comprises: generating an alarm based on the identified patient-status.

12. A method for determining pain level in a clinical environment comprising:
monitoring continuously at least one of facial expressions and sound generated by a patient;
analyzing the at least one of facial expressions and sound for determining the pain level; and
translating the at least one of facial expressions and sound to a quantifiable parameter indicating the pain level.

13. A method as in claim 12, wherein the step of monitoring comprises: providing a three dimensional imager for continuously recording the at least one of facial expressions and sound generated by the patient.

14. A method as in claim 12, wherein the step of analyzing further comprises: verifying the authenticity of the at least one of facial expressions and sound generated by the patient.

15. A method as in claim 12, wherein the step of translating comprises: deriving a pain-value, from the facial expressions.

16. A method as in claim 15, wherein the step of translating comprises: deriving a pain-value, from the sound generated by the patient.

17. A method as in claim 16, wherein the step of translating comprises: deriving a pain level from the pain-values obtained from the at least one of facial expressions and the sound.

18. A method as in claim 12, wherein the method further comprises: electronically recording the pain level at a given instance corresponding to a patient.

19. A method as in claim 18, wherein the step of recording further comprises: generating an alarm based on the identified pain level.

20. An automatic pain recording system comprising:
a detector for continuously monitoring gestures of a patient including facial expressions and sound generated by the patient;
an analyzer coupled to the detector for analyzing the gestures for identifying a pain level; and
a recorder for recording the identified pain level.

21. A system as in claim 20, wherein the detector is a video imager.

22. A system as in claim 20, wherein the analyzer includes a processor and a translator.

23. A system as in claim 20, wherein the recorder includes an indicator.
